(19) United States
(12) Patent Application Publication
(10) Pub. No.: US 2023/0181909 A1
(43) Pub. Date: Jun. 15, 2023

(54) SYSTEMS FOR USING LOCAL FIELD POTENTIAL OSCILLATIONS

(71) Applicant: Boston Scientific Neuromodulation Corporation, Valencia, CA (US)

(72) Inventors: Rosana Esteller, Santa Clarita, CA (US); Tianhe Zhang, Studio City, CA (US)

(21) Appl. No.: 18/075,647

(22) Filed: Dec. 6, 2022

Related U.S. Application Data
(60) Provisional application No. 63/287,661, filed on Dec. 9, 2021.

(51) Int. Cl. A61N 1/36; A61N 1/05

(52) U.S. Cl. CPC A61N 1/36139; A61N 1/36146; A61N 1/0553

(57) ABSTRACT

A system may include a neuromodulator, a local field potential sensor, a feature extractor, a comparator and control circuitry. The neuromodulator may be configured to use neuromodulation parameters to deliver a neuromodulation signal to neural tissue in or near a spinal cord. The LFP sensor may be configured to sense local field potentials within a spinal cord or a peripheral nerve that are indicative of spinal cord oscillations. The feature extractor may be configured to extract one or more features for the local field potentials indicative of the spinal cord oscillations. The comparator may be configured to provide a comparison of the one or more extracted features to corresponding one or more setpoints. The control circuitry may be configured to control the delivery of the neuromodulation signal based on the comparison.

### CLAIM OF PRIORITY

This application claims the benefit of U.S. Provisional Application No. 63/287,661, filed on Dec. 9, 2021, which is hereby incorporated by reference in its entirety.

### TECHNICAL FIELD

This document relates generally to medical systems, and more particularly, but not by way of limitation, to systems, devices, and methods for using oscillations within sensed local field potentials (LFPs) in the spinal cord or peripheral nerves (e.g., autonomic nerves) or neural structures in the peripheral nervous system for neurological disorders.

### BACKGROUND

Therapy devices are devices configured to deliver a therapy. These devices may be external or implantable. Examples of therapy devices include electrical therapy devices such as neuromodulators and cardiac rhythm management devices, mechanical therapy devices, thermal therapy devices, and drug delivery devices. Examples of neuromodulators include, but are not limited to, spinal cord stimulators (SCS), deep brain stimulators (DBS), peripheral nerve stimulation (PNS) and function electrical stimulation (FES). Examples of cardiac rhythm management device include, but are not limited to, pacemakers and defibrillators. Examples of mechanical devices include, but are not limited to, devices configured to deliver compression to prevent deep vein thrombosis or to massage fluid from legs. Examples of drug delivery devices include, but are not limited to, insulin pumps or other infusion pumps.

With respect to neuromodulators, for example, an external programming device may be used to program the implantable neurostimulator with modulation parameters controlling the delivery of the neuromodulation energy. For example, modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y).

Conventionally, the customization of values for these parameters to a patient can be very time costly. For example, the modulation parameters may be configured as a neuromodulation program capable of being implemented by the neuromodulator, and the neurostimulator may be programmed with more than one program. In order to find a program that provides an effectively provides a therapy (e.g., pain relief) with negligible side effects, the patient or clinician may implement different programs within the neuromodulator.

Sensing electrophysiological data while providing a therapy provides a plausible closed-loop feedback mechanism by which to regulate the therapy. For example, closed-loop algorithms for SCS or PNS or autonomic nerve stimulation may use extracted features from a biological system to update therapy. However, there is a need to improve closed-loop control to regulate the therapy.

### SUMMARY

An example (e.g., “Example 1”) of a system may include a neuromodulator, a local field potential (LFP) sensor, a feature extractor, a comparator and control circuitry. The neuromodulator may be configured to use neuromodulation parameters to deliver a neuromodulation signal to neural tissue. The LFP sensor may be configured to sense local field potentials within a spinal cord or a peripheral nerve that are indicative of oscillations. The feature extractor may be configured to extract one or more features for the local field potentials indicative of the oscillations. The comparator may be configured to provide a comparison of the one or more extracted features to corresponding one or more setpoints. The control circuitry may be configured to control the delivery of the neuromodulation signal based on the comparison. The LFP may be activity that is occurring without stimulation, but whose change is modulated with stimulation. For example, the sensed LFPs may be in the spinal cord or peripheral nerves (e.g., autonomic nerves) or neural structures in the peripheral nervous system. For example, the neuromodulation signal may be delivered to tissue to provide a therapy such as SCS, PNS, or autonomic neuromodulation.

In Example 2, the subject matter of Example 1 may optionally be configured such that the feature extractor is configured to extract at least one time domain feature used by the comparator to provide the comparison.

In Example 3, the subject matter of Example 2 may optionally be configured such that the at least one time domain feature includes at least one of: peak to peak amplitude, standard deviation vs. mean, oscillation frequency, variance of peak-to-peak times, variance of the individual min-max ranges, area under the curve (AUC), curve length, RMS amplitude, a regression measure of drift over time, or a measure of power.

In Example 4, the subject matter of any one or more of Examples 1-3 may optionally be configured such that the feature extractor is configured to extract at least one frequency domain feature or wavelet domain feature used by the comparator to provide the comparison.

In Example 5, the subject matter of Example 4 may optionally be configured such that the at least one frequency domain feature includes at least one of: signal overall power in passband or at specific bands, max peaks within specific target bands, width of peak, standard deviation of height of X most prominent peaks, or an area under a curve of a maximum peak.

In Example 6, the subject matter of any one or more of Examples 1-5 may optionally be configured such that the corresponding one or more setpoints include at least one feature for the local field potentials indicative of oscillations corresponding to a symptom level, a therapy rating or side-effect ratings.

In Example 7, the subject matter of any one or more of Examples 1-6 may optionally be configured such that the control circuitry is configured to provide a feedback closed loop control using a Proportion Integral Derivative (PID), PID with thresholds, a lookup table, a Kalman control, an On/Off control or a threshold control.

In Example 8, the subject matter of any one or more of Examples 1-7 may optionally be configured such that the electrodes are designed, placed or orientated to enhance sensing of oscillations in a spinal cord, wherein the electrodes include: electrodes arranged in a paddle array and rostrocaudally orientated; cylindrical electrodes with a large diameter or large surface area to increase sensing surface; intradural electrodes; epidural electrodes; or electrodes placed over the dorsal horn.

In Example 9, the subject matter of any one or more of Examples 1-8 may optionally be configured to further include at least one of: a filter configured to filter out at least one of noise, ECAPs or one or more artifacts from the sensed local field potentials; or a filter configured to filter frequencies of interest from the sensed local field potentials

In Example 10, the subject matter of any one or more of Examples 1-9 may optionally be configured to further include other sensors to provide at least one other sensor signal, wherein the feature extractor is configured to extract at least one other feature from the other sensor signal, the comparator is configured to provide a comparison of the at least one other feature from the other sensor signal to at least one other setpoint, and the control circuitry is configured to control the delivery of the neuromodulation signal based on both the comparison of the extracted features to corresponding setpoints and the comparison of the extracted at least one other feature from the other sensor signal to the at least one other setpoint.

In Example 11, the subject matter of any one or more of Examples 1-10 may optionally be configured such that the system includes at least one processor configured to provide machine learning to classify the corresponding one or more setpoints from learning data, wherein the learning data is gathered using intervals of stimulation and recording, wherein the intervals between stimulation are determined using known stimulation onset/offset times, patient preference, or a signal duration of sufficient length to obtain a desired amount of learning data.

In Example 12, the subject matter of any one or more of Examples 1-10 may optionally be configured such that the system includes at least one processor configured to provide machine learning to classify the corresponding one or more setpoints from learning data, wherein the one or more setpoints correspond to a state based on a quantitative mapping of features of the for the local field potentials that are indicative of the oscillations for: baseline and therapy; qualitative based on a pain score; or overlaid upon pre-defined datasets.

In Example 13, the subject matter of any one or more of Examples 1-10 may optionally be configured such that the system includes at least one processor configured to provide machine learning to classify the corresponding one or more setpoints from learning data, and to map relationships between correlated features and states, wherein the mapped relationships include: a regressive fit for each state to determine the relationships between the corresponding state and features; or a plot for a ratio of correlated variables against programming parameters.

In Example 14, the subject matter of any one or more of Examples 1-10 may optionally be configured such that the system includes at least one processor configured to provide machine learning to classify the corresponding one or more setpoints from learning data, including comparing multiple states and their features to determine dState/(dSCS or dPNS or dANS), where dState/(dSCS or dPNS or dANS) represents a change in a state with respect to a change in an spinal cord stimulation (SCS) parameter or a peripheral nerve stimulation (PNS) parameter or an autonomic nerve stimulation (ANS) parameter, and performing a sensitivity analysis on a plurality of parameters to identify one or more of the plurality of parameters having a greater change between bad and good states with a change in SCS, PNS or ANS parameter than other ones of the plurality of parameters, wherein the controlling the delivery of the neuromodulation signal based on the comparison includes modulating the one or more of the plurality of parameters based on the comparison.

In Example 15, the subject matter of any one or more of Examples 1-10 may optionally be configured such that the system includes at least one processor configured to provide machine learning to classify the corresponding one or more setpoints from learning data, including use a neural network, a Support Vector Machine (SVM), a least square model, or a mean squares model to determine how state variables change with stimulation, for use in controlling the delivery of the neuromodulation signal.

The processor(s) referenced in any two or more Examples 11-15 may be the same processor(s) to perform the corresponding two or more processes in the corresponding examples.

Example 16 includes subject matter (such as a method, means for performing acts, machine readable medium including instructions that when performed by a machine cause the machine to performs acts, or an apparatus to perform). The subject matter may include: delivering a neuromodulation signal according to neuromodulation parameters to neural tissue; sensing local field potentials within a spinal cord or a peripheral nerve indicative of oscillations; extracting one or more features from the local field potentials that are indicative of the oscillations; providing a comparison of the extracted one or more feature(s) to corresponding one or more setpoints; and controlling the delivery of the neuromodulation signal based on the comparison. The local field potentials may be activity that is occurring without stimulation, but whose change is modulated with stimulation. For example, the sensed local field potentials may be in the spinal cord or peripheral nerves or neural structures in the peripheral nervous system. For example, the neuromodulation signal may be delivered to tissue to provide a therapy such as SCS, PNS, or autonomic neuromodulation.

In Example 17, the subject matter of Example 16 may optionally be configured such that the extracting one or more features includes extracting at least one of a time domain feature, a frequency domain feature or a wavelet domain feature.

In Example 18, the subject matter of Example 16 may optionally be configured such that the extracting one or more features include the extracting at least one time domain feature.

In Example 19, the subject matter of Example 18 may optionally be configured such that the extracting at least one time domain feature includes extracting at least one of: peak to peak amplitude, standard deviation vs. mean, oscillation frequency, variance of peak-to-peak times, variance of the individual min-max ranges, area under the curve (AUC), curve length, RIVIS amplitude, a regression measure of drift over time, or a measure of power.

In Example 20, the subject matter of any one or more of Examples 16-19 may optionally be configured such that the extracting one or more features includes the extracting at least one frequency domain feature.

In Example 21, the subject matter of Example 20 may optionally be configured such that the extracting at least one frequency domain feature includes extracting at least one of: signal overall power in passband or at specific bands, max peaks within specific target bands, width of peak, standard deviation of height of X most prominent peaks, or an area under a curve of a maximum peak.

In Example 22, the subject matter of any one or more of Examples 16-21 may optionally be configured such that the corresponding one or more setpoints include at least one feature for the local field potentials indicative of oscillations corresponding to a symptom level, a therapy rating or side-effect ratings.

In Example 23, the subject matter of any one or more of Examples 16-22 may optionally be configured such that the controlling the delivery of the neuromodulation signal based on the comparison includes providing a feedback closed loop control using a Proportion Integral Derivative (PID), PID with thresholds, a lookup table, a Kalman control, an On/Off control or a threshold control.

In Example 24, the subject matter of any one or more of Examples 16-23 may optionally be configured such that the sensing local field potentials indicative of spinal cord oscillations includes using electrodes designed, placed or orientated to enhance sensing of spinal cord oscillations.

In Example 25, the subject matter of Example 24 may optionally be configured such that the electrodes include: electrodes arranged in a paddle array and rostrocaudally orientated; cylindrical electrodes with a large diameter or large surface area to increase sensing surface; intradural electrodes; epidural electrodes; or electrodes placed over the dorsal horn.

In Example 26, the subject matter of any one or more of Examples 16-25 may optionally be configured to include filtering the sensed local field potentials to filter out at least one of noise, ECAPs, or one or more artifacts, or performing bandpass filtering frequencies of interest.

In Example 27, the subject matter of any one or more of Examples 16-26 may optionally be configured to further include using at least one other sensor to provide at least one other sensor signal, extracting at least one other feature from the other sensor signal, and providing a comparison to the at least one other feature from the other sensor signal to at least one other setpoint.

In Example 28, the subject matter of any one or more of Examples 16-27 may optionally be configured to include using machine learning to evaluate learning data to classify the one or more setpoints.

In Example 29, the subject matter of Example 28 may optionally be configured such that the using machine learning includes using a neural network, a Support Vector Machine (SVM), a least square model, or a mean squares model to determine how state variables change with stimulation, for use in controlling the delivery of the neuromodulation signal.

In Example 30, the subject matter of any one or more of Examples 16-29 may optionally be configured to further include gathering learning data using intervals of stimulation and recording, wherein the intervals between stimulation are determined using known stimulation onset/offset times, patient preference, or a signal duration of sufficient length or with sufficient delay to obtain a desired amount of learning data with appropriate delay.

In Example 31, the subject matter of any one or more of Examples 16-30 may optionally be configured such that the one or more setpoints correspond to a state based on a quantitative mapping of features for the local field potentials that are indicative of the spinal cord oscillations for: baseline and therapy; qualitative based on a pain score; or overlaid upon pre-defined datasets.

In Example 32, the subject matter of any one or more of Examples 16-31 may optionally be configured to further include mapping relationships between correlated features and states, wherein the mapping relationships includes: performing a regressive fit for each state to determine the relationships between the corresponding state and features; or plotting a ratio of correlated variables against programming parameters.

In Example 33, the subject matter of any one or more of Examples 16-32 may optionally be configured to further include comparing multiple states and their features to determine dState/(dSCS or dPNS or dANS), where dState/(dSCS or dPNS or dANS) represents a change in a state with respect to a change in an spinal cord stimulation (SCS) parameter or a peripheral nerve stimulation (PNS) parameter or an autonomic nerve stimulation (ANS) parameter; and performing a sensitivity analysis on a plurality of parameters to identify one or more of the plurality of parameters having a greater change between bad and good states with a change in SCS, PNS or ANS parameter than other ones of the plurality of parameters, wherein the controlling the delivery of the neuromodulation signal based on the comparison includes modulating the one or more of the plurality of parameters based on the comparison.

In Example 34, the subject matter of any one or more of Examples 16-33 may optionally be configured such that the one or more setpoints corresponds to a preconfigured state determined based on a patient's diagnosis or other demographic factors, or correspond to a user-customizable state.

Example 35 includes subject matter (such as a device, apparatus, or machine) that may include non-transitory machine-readable medium including instructions, which when executed by a machine, cause the machine to perform a method comprising delivering a neuromodulation signal according to neuromodulation parameters to neural tissue, sensing local field potentials within a spinal cord or a peripheral nerve indicative of oscillations, extracting one or more features for the local field potentials that are indicative of the oscillations, providing a comparison of the extracted one or more features to corresponding one or more setpoints, and controlling the delivery of the neuromodulation signal based on the comparison. The local field potentials may be activity that is occurring without stimulation, but whose change is modulated with stimulation. For example, the sensed local field potentials may be in the spinal cord or peripheral nerves or neural structures in the peripheral nervous system. For example, the neuromodulation signal may be delivered to tissue to provide a therapy such as SCS, PNS, or autonomic neuromodulation.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a therapy system.

FIG. 2 illustrates, by way of example and not limitation, the neuromodulation system of FIG. 1 implemented in a spinal cord stimulation (SCS) system.

FIG. 3 illustrates, by way of example and not limitation, a modulation device capable of selecting features of a sensed electrical signal for use to provide feedback for closed-loop control.

FIG. 4 illustrates, by way of example and not limitation, a system configured to provide closed-loop adjustment of a therapy based on detected oscillations in sensed local field potentials (LFPs) in the spinal cord.

FIG. 5 illustrates, by way of example and not limitation, a system configured to provide closed-loop adjustment of a therapy based on detected oscillations in sensed LFPs in the spinal cord.

FIG. 6 illustrates, by way of example and not limitation, methods for providing closed-loop adjustment of a therapy based, at least in part, on both detected oscillations in sensed LFPs in the spinal cord or peripheral nerves (e.g., autonomic nerves).

FIG. 7 illustrates, by way of example and not limitation, systems for detecting oscillation signals in sensed LFP signals for further analysis to extract features from the oscillations.

FIG. 8 illustrates, by way of example and not limitation, training and use of a machine-learning program.

FIG. 9 illustrates, by way of example and not limitation, a process for determining state(s), feature value(s) for state(s) and relationships between state(s) and feature(s) from learning (or training) data.

FIG. 10 illustrates, by way of example and not limitation, learning data that may be used to provide the machine-learning program training.

FIG. 11 illustrates, by way of example and not limitation, quantitative mapping of metrics defining oscillations to qualitative state such as patient pain, other symptoms levels, therapy ratings, side-effect ratings, and the like.

FIG. 12 illustrates, by way of example and not limitation, quantitative mapping of metrics defining oscillations overlaid on pre-defined data sets to identify a patient disease state.

FIG. 13 illustrates, by way of example and not limitation, the use of regression fits to identify relationships between state(s) and some extracted feature(s) from the oscillation signal when the states are correlated.

FIG. 14 illustrates, by way of example and not limitation, variables plotted as a ratio against a parameter and/or programming settings (“Stim P1” and Stim P2″).

FIG. 15 illustrates, by way of example and not limitation, epidural oscillation signals in the time domain representing a healthy state, a disease state and effective spinal cord stimulation.

FIG. 16 illustrates a sensitivity plot identifying parameter(s) most or least different between good and bad states.

FIG. 17 illustrates an example in which the stimulation parameters (amplitude, pulse width, frequency or rate, and pattern) are assessed for their ability to modulate the extracted feature(s) determined to be most sensitive to changes between the good and bad states.

FIG. 18 illustrates, by way of example and not limitation, selection of a parameter for use in controlling the therapy based on a-priori knowledge of disease state.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Aspects of the present subject matter relate to treating neurological conditions by sensing local oscillations in the spinal cord or peripheral nerves or nerve structures like the dorsal roots, dorsal root ganglia (DRG), or targets in the peripheral (e.g., autonomic) nervous system. For simplicity in the disclosure references are made to the spinal cord, but they could be considered for any nerve or neural structure in the peripheral nervous system. For example, extracted feature(s) from the sensed local oscillations in the spinal cord may provide biomarker(s) that can be used as an objective replacement for subjective paresthesia for programming neuromodulation. Spontaneous oscillations are typical in the nervous system, and they suggest network communications between different neural structures in the neural circuit. These oscillations are typically observed in gray matter and therefore are harder to detect in the spinal cord as the white matter tracts are more superficially located. These oscillations are expected to depend on the SCS stimulation parameters used and on the chronic pain stage, and can be used as a biomarker of therapy response for use in a closed-loop SCS system.

There is a distinction between oscillations and evoked potentials. Evoked potentials result in a direct summation of immediate, synchronized response to stimulation. Evoked responses depend on stimulation occurring, as they are always evoked by stimulus (electrical, sensory stimulus, sound piezoelectic, photobiomodulation-light, thermal stimulation). Evoked potentials relate or originate from white matter (axonal activity). Oscillations result from indirect, second+ order and dispersed/diffuse activity of neural circuit. The oscillations may still change in response to stimulation, but may also be apparently uncorrelated and may occur in absence of stimulation. For example, oscillations are present in natural and/or physiological background activity. Various aspects of the present subject matter focus on using oscillations, which may be spontaneous or evoked and may relate or originate from gray matter (neuronal activity).

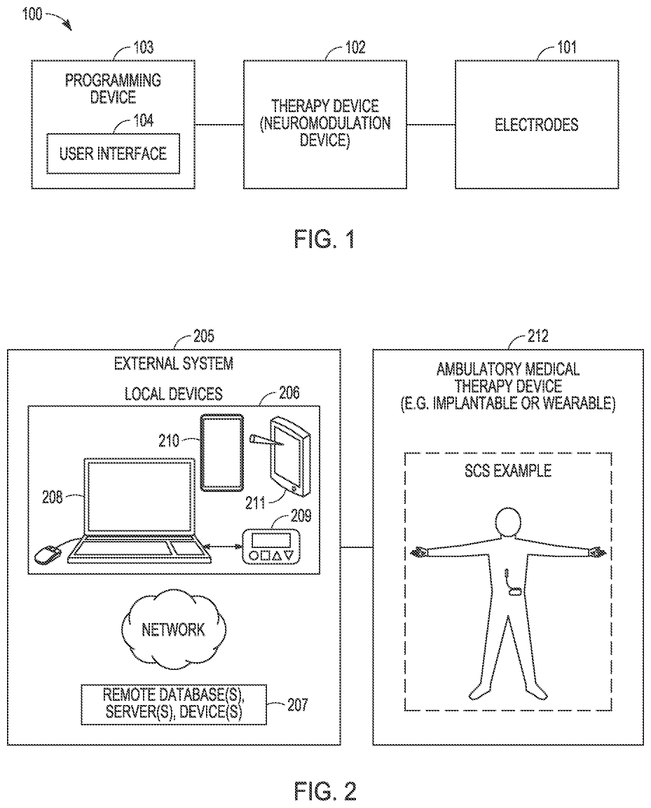

FIG. 1 illustrates, by way of example, an embodiment of a therapy system. The therapy system includes a therapy device configured to deliver a therapy. The therapy device may be an implantable device or an external device, including wearable devices. Examples of therapy devices include electrical therapy devices such as neuromodulators and cardiac rhythm management devices, mechanical therapy devices, thermal therapy devices, and drug delivery devices. Examples of neuromodulators include, but are not limited to, spinal cord stimulators (SCS), deep brain stimulators (DBS), peripheral nerve stimulation (PNS) and functional electrical stimulation (FES). This disclosure discusses SCS systems as an example of a therapy device, but is applicable to PNS or autonomic nerve stimulation (ANS).

More particularly, FIG. 1 illustrates a neuromodulation system 100 that includes electrodes 101, a neuromodulation device 102 and a programming system such as a programming device 103, which may be or may include a clinician programmer. The programming system may include multiple devices that may be configured to communicate with each other (e.g., remote control, clinician programmer, phone, tablet, and the like). The electrodes 101 are configured to be placed on or near one or more neural targets in a patient. The neuromodulation device 102 is configured to be electrically connected to electrodes 101 and deliver neuromodulation energy, such as in the form of electrical pulses or other waveform, to the one or more neural targets though electrodes 101. The system may also include sensing circuitry to sense a biological signal, which may but does not necessarily form a part of neuromodulation device 102. The sensed biological signal may include, but are not limited to, sensed LFPs in the spinal cord or a peripheral nerve, such as an autonomic nerve, from which oscillations may be detected. Other biological signals may be sensed. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver. For example, the parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude, pulse width, pulse rate, and burst rate. The programming device 103 enables the user to access the user-programmable parameters, and may also provide the user with data indicative of the sensed biological signal or feature(s) of the sensed biological signal. In various embodiments, the programming device 103 is configured to be communicatively coupled to modulation device via a wired or wireless link. In various embodiments, the programming device 103 includes a user interface 104 such as a graphical user interface (GUI) that allows the user to set and/or adjust values of the user-programmable modulation parameters. The user interface 104 may also allow the user to view the data indicative of the sensed biological signal or feature(s) of the sensed biological signal and may allow the user to interact with that data. For example, a user may be able to provide some input used to weight or otherwise relate the data for use to provide a control algorithm for delivering the therapy. The neuromodulation device 102, the programming device 103 and other devices or system may collect data that may be used by the neuromodulation system 100. For example, the user interface 104 may be used to allow the user to answer healthcare-related questions, such as but not limited to the efficacy of the therapy.

The therapy device may provide a closed-loop therapy, in which sensing circuitry is configured for use to detect a biological signal for use to provide feedback. Sensing circuitry may include various components such as an application specific integrated circuit (ASIC), hardware and/or firmware. Sensing circuitry may include software implemented using a processor to further analyze feature(s) of the biological signal. The biological signal may be a measurable signal produced by electrical, chemical or mechanical activity. Examples of electrical signals may include sensing electrical activity in the brain (e.g., EEGs), electrical activity in nerves and muscles (e.g., EMGs), cardiac activity (e.g., ECGs), and other nerve sensing including both non-evoked responses and evoked responses (e.g., evoked compound action potentials (ECAPs), local evoked potentials (LEPs), and/or evoked resonant nerve activity (ERNA)). Examples of mechanical signals may include sounds contractions detected via flex or strain sensors. Examples of chemical signals may include detected analyte concentrations such as glucose and the like. The system may include a feature detector that is configured to detect a plurality of available features of the biological signal. At least one of the features may be used as a closed-loop sensed feature of the biological signal, which may be used by a controller to provide a closed-loop therapy. The closed-loop sensed feature may be compared to a setpoint of that feature, and the difference may be fed into a feedback control algorithm. Control circuitry within the therapy device may be used to control the therapy based on the sensed biological signal(s). The control circuitry may include a controller/processor and/or may include other hardware, firmware and/or software.

FIG. 2 illustrates, by way of example and not limitation, the neuromodulation system of FIG. 1 implemented in a spinal cord stimulation (SCS) system. The illustrated neuromodulation system 200 includes an external system 205 that may include at least one programming device. The illustrated external system 205 may include device(s), at least some of which are configured for use by a clinician to communicate with and program an ambulatory medical therapy device, such as an implantable neuromodulator. Examples of the neuromodulator may include a spinal cord stimulator. The external system 205 may include a network of computers, including local device(s) 206 and remote systems 207. The local device(s) 206 may include one or more of a programmer 208, a remote control 209, a phone 210 (e.g., smartphone), and a tablet 211. The remote system 207 may include remote database(s), remote server(s) and remote device(s) capable of communication through the network to local device(s). The external system 205 may be configured for use to program or receive data from the ambulatory therapy device 212. For example, the clinical programmer 208 may be used to program the therapy device 212, using input from remote devices 207 or feedback received from other local devices 206. By way of example and not limitation, the remote control device 207 may allow the patient to turn a therapy on and off and/or may allow the patient to adjust patient-programmable parameter(s) of the plurality of modulation parameters, and the phone 210 or tablet 211 may be used to answer questionnaires to provide input for therapy control. FIG. 2 illustrates a neuromodulation device 202 as an implantable device, although a neuromodulation device 202 may be an external device such as a wearable device. The external system 205 may include a wearables such as a watch, sensors or therapy-applying devices. The watch may include sensor(s), such as sensor(s) for detecting activity, motion and/or posture. Other wearable sensor(s) may be configured for use to detect various physiological parameters such as, but not limited to, activity, motion and/or posture of the patient that may be useful input to control the therapy delivery.

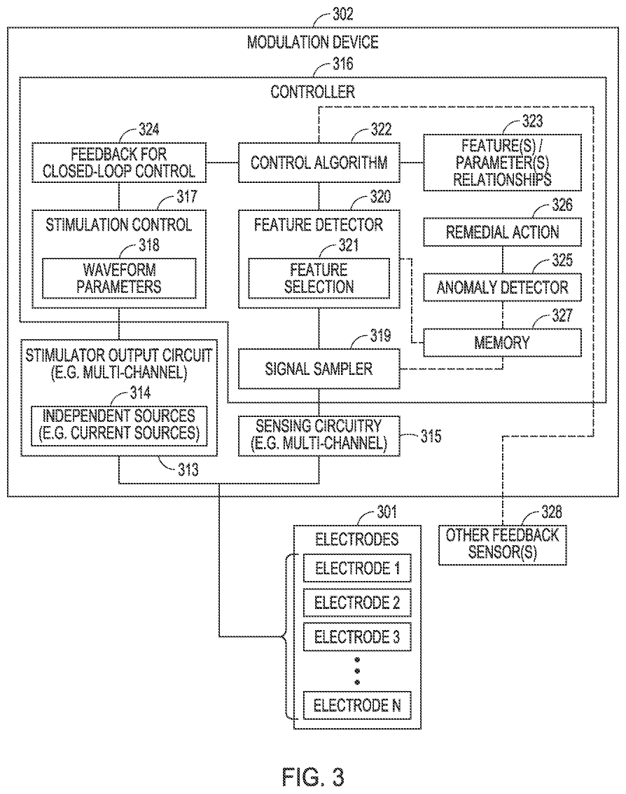

FIG. 3 illustrates, by way of example and not limitation, a modulation device capable of selecting features of a sensed electrical signal for use to provide feedback for closed-loop control. The modulation device 302 is an example of a therapy device illustrated in FIG. 1, and may be configured for use to deliver SCS therapy, for example. The modulation device 302 may be configured to be connected to electrode(s) 301, illustrated as N electrodes. Any one or more of the electrodes 301 may be configured for use to deliver modulation energy, sense electrical activity, or both deliver modulation energy and sense electrical activity. The modulation device 302 may include a stimulator output circuit 313 configured to deliver modulation energy to electrode(s). The stimulator output circuit 313 may be configured with multiple (e.g., two or more) channels for delivering modulation energy, where each channel may be independently controlled with respect to other channel(s). For example, the stimulator output circuit 313 may have independent sources 314 such as independent current sources or independent voltage sources. The modulation device 302 may include sensing circuitry 315 configured to receive sensed electrical energy from the electrode(s), such as may be used to sense local field potentials in the spinal cord and/or sense other electrical activity in neural tissue or muscle tissue. The sensing circuitry 315 may be configured to process signals in multiple (e.g., two or more) channels. By way of example and not limitation, the sensing circuitry 315 may be configured to amplify and filter the signal(s) in the channel(s). Additionally or alternatively, the sensing circuitry may be configured for use with other types physiological sensors, such as but not limited to activity, posture, respiration, heart rate, electrocardiogram (ECG), oxygen level, strain sensors, or analyte sensors.

The modulation device 302 may include a controller 316 operably connected to the stimulator output circuit 313 and the sensing circuitry 315. The controller 316 may include a stimulation control 317 configured for controlling the stimulator output circuit 313. For example, the stimulation control 317 may include start/stop information for the stimulation and/or may include relative timing information between stimulation channels. The stimulation control 317 may include waveform parameters 318 that control the waveform characteristics of the waveform produced by the stimulator output circuit 313. The waveform parameters 318 may include, by way of example and not limitation, amplitude, frequency, and pulse width parameters. The waveform parameters 318 may include, by way of example and not limitation, regular patterns such as patterns regularly repeat with same pulse-to-pulse interval and/or irregular patterns of pulses such as patterns with variable pulse-to-pulse intervals. The waveform parameters 318 may, but do not necessary, define more than one waveform shape (e.g., including a shape other than square pulses with different widths or amplitudes). The stimulation control 317 may be configured to change waveform parameter(s) (e.g., one or more waveform parameters) in response to user input and/or automatically in response to feedback.

The controller 316 may include a signal sampler 319 configured for use to sample a signal produced by the sensing circuitry 315. The controller 316 may further include a feature detector 320 configured to detect one or more features in the sampled signal. A few examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Low-level hardware such as ASICs may extract some features and higher-level hardware/firmware may detect other features through board-level sensors and code. Firmware updates may be used to enable other features to be detected. Software may also be used to detect features. The feature detector may include a feature selection 321 for determining or otherwise providing the selected closed-loop sensed feature. Detected feature(s) from the feature detector 321 may be fed into a control algorithm 322, which may use relationship(s) 323 between the feature(s) and waveform parameter(s) to determine feedback for closed-loop control 324 of the therapy. By way of example, these relationships may be determined using machine learning processes and training data. More than one algorithm may be used to provide the closed-loop control. The algorithm(s) may be selected from a plurality of algorithms that are available to be used to implement the closed-loop control. The different algorithms may use different feature(s) and/or control different waveform parameter(s), and/or have different transfer functions or sensitivity for adjusting the parameter(s) in response to changes in the feature(s). The closed-loop control 324 may be used by the stimulation control 317 to adjust the stimulation (e.g., parameter(s)). The controller 316 of the modulation device 302 may further include an anomaly detector 325 configured to detect anomalies in the feature(s) detected by the feature detector 320. These anomalies may be detected based on the feature data (e.g., training data) used to determine the relationship(s) 323 between the feature(s) and the waveform parameter(s). The controller 316 of the modulation device 302 may further be configured to perform at least some activities for providing remedial action 326 in response to a detected anomaly or detected anomalies. The remedial action may be transitioning from a closed-loop therapy to an open loop therapy, or stopping therapy. The controller 316 may include a memory 327 for storing the detected feature(s) and/or storing the sampled signals, for analysis (e.g., retrospective analysis). The illustrated modulation device 302 may also receive feedback from other sensors 328 as in input into a control algorithm 322 for use to provide feedback for closed-loop control 324.

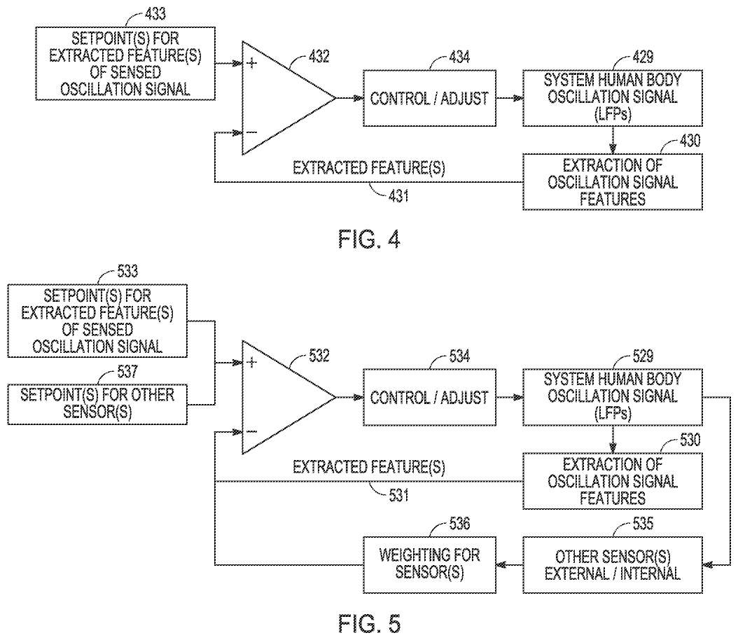

FIG. 4 illustrates, by way of example and not limitation, a system configured to provide closed-loop adjustment of a therapy based on detected oscillations in sensed LFPs. The LFPs may be sensed using implanted electrodes, such as electrodes positioned in the epidural space to detect LFPs in the spinal cord. The illustrated system may provide continuous adjustment based on learning from an initial clinic visit.

The spinal cord oscillation signal or peripheral nerve oscillation signal may be detected in sensed LFPs, as generally illustrated at 429, and feature(s) of the oscillation signal may be extracted at 430. The sensed oscillation signal undergoes a feature extraction process to determine specific features from the signal such as, but not limited to, power in frequency bands, peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline., zero crossings, and the like. The signal may be characterized by longer term oscillations, with respect to signals in the brain, that may or may not be related to the stimulus. These extracted feature(s) 431 may be compared using comparator 432 to setpoint(s) for the extracted feature(s) 433. The therapy may be controlled or adjusted at 434 based on the comparison.

The feedback control may use various techniques, such as Proportional, Integral, Derivative (PID), a PID with thresholds, a lookup table, a neural network, SVM, a linear mean squares model (including least square model or a means squares model), a Kalman control, a simple On/Off control or a threshold control. Regarding PID, the proportional component may depend only on an error signal, representing a difference between the set point and the process variable, where a proportion gain represents a ratio of an output response to the error signal. An integral component sums the error term over time, and a derivative component causes the output to decrease if the process variable is increasing rapidly. A neural network may include an input node layer, an output node layer, and hidden layer(s) in between the input and output. The nodes may be weighted. An output of a node that is above a threshold will be activated to send data to the next layer of the network. A probabilistic neural network (PNN) may be used to handle classification and pattern recognition problems, and a K-Nearest neighbors (KNN) may be used to classify data with respect to number of nearest distance to system training data KNN. A Support Vector Machine (SVM) may be used to distinctly classify data by finding a plane that separates the classes. A Kalman control uses a series of measurements observed over time to produce estimates of unknown variables that tend to be more accurate than those based on a single measurement alone.

The sensed spinal cord or peripheral nerve oscillation signal undergoes a feature extraction process to determine specific features from the signal such as power in frequency bands, range, curve length, AUC, zero crossings, and the like. Of note, the signal is characterized by longer term oscillations that may or may not related to the stimulus in a similar way as they occur in the brain (e.g., beta peak present in when PD symptoms like tremor are not well controlled, reduction of beta peak when stimulation is applied in the appropriate gray matter target like subthalamic nucleus (STN).

FIG. 5 illustrates, by way of example and not limitation, a system configured to provide closed-loop adjustment of a therapy based on detected oscillations in sensed LFPs in the spinal cord. Components 529, 530, 531, 532, 533 and 534 in FIG. 5 are similar to 429, 430, 431, 432, 433 and 434 are similar to components in FIG. 4. Furthermore, the system may include other internal or external sensor(s) 535 configured to sense biological signal(s) from the body. The system may weight, as illustrated at 536, the data sensed by these other sensor(s). The comparator 532 may compare the weighted data 536 from the sensor(s) to the setpoint(s) 537 for the other sensor(s) to provide another error to control or adjust the therapy.

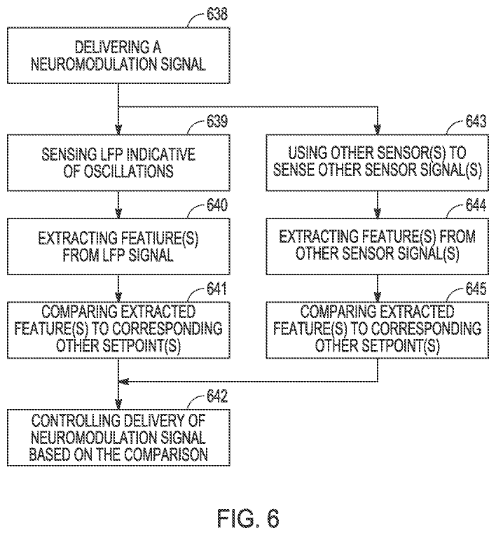

FIG. 6 illustrates, by way of example and not limitation, methods for providing closed-loop adjustment of a therapy based, at least in part, on both detected oscillations in sensed LFPs in the spinal cord or peripheral nerves. Peripheral nerves include autonomic nerves, and some embodiments may detect oscillations in sensed LFPs in an autonomic nerve. A method may include delivering a neuromodulation signal according to neuromodulation parameters to neural tissue in or near a spinal cord or a peripheral nerve depending on the specific case 638; sensing local field potentials indicative of oscillations, such as spinal cord oscillations or a peripheral nerve oscillations (e.g., an autonomic nerve oscillations) 639; extracting one or more features from the local field potentials that are indicative of the oscillations 640; providing a comparison of the extracted one or more feature(s) to corresponding one or more setpoints 641; and controlling the delivery of the neuromodulation signal based on the comparison 642. The extracted feature(s) 640 may be a time domain feature, a frequency domain feature and/or a wavelet domain feature. Examples of time domain feature(s) that may be extracted include, but are not limited to, peak to peak amplitude, standard deviation vs. mean, oscillation frequency, variance of peak-to-peak times, variance of the individual min-max ranges, area under the curve (AUC), curve length, RIVIS amplitude, a regression measure of drift over time, or a measure of power. Examples of frequency domain feature(s) that may be extracted include, but are not limited to, signal overall power in passband or at specific bands, max peaks within specific target bands, width of peak, standard deviation of height of X most prominent peaks, or an area under the curve of the maximum peak. The setpoint(s) may include at least one feature for the local field potentials indicative of oscillations corresponding to a symptom level, a therapy rating or side-effect ratings. The control of the delivery of neuromodulation 642 may include providing a feedback control using a Proportion Integral Derivative (PID), a lookup table, a Kalman control, an On/Off control or a threshold control. The delivery of neuromodulation 642 may also be controlled based on using other sensor(s) to sense other sensor signal(s) 643, extracting feature(s) from the other sensor signal(s) 644, and comparing extracted feature(s) to corresponding other setpoint(s) 645.

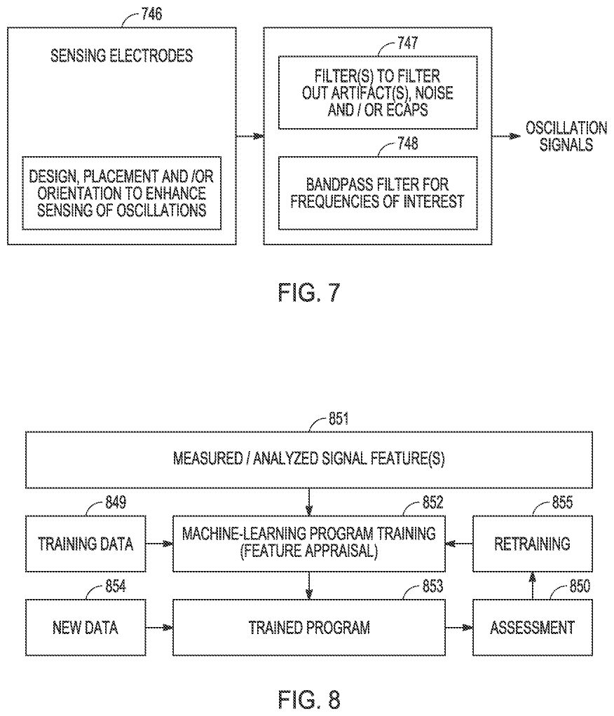

FIG. 7 illustrates, by way of example and not limitation, systems for detecting oscillation signals in sensed LFP signals for further analysis to extract features from the oscillations. Sensing spinal oscillations poses a challenging for regular epidural electrodes. The present subject matter includes various techniques to enhance the ability to sense spinal oscillations. For example, the sensing electrodes may be designed, placed, and/or oriented to enhance the sensing of oscillations. The oscillations in the spinal cord may be sensed using a paddle array oriented rostrocaudally increasing the diameter of the cylindrical contacts or otherwise increasing the surface area (to increase sensing surface), using intradural electrodes, using epidural electrodes, optimizing placement for automatic sensing of the local field potentials (e.g., placing electrodes laterally over dorsal horn), using filters 747 to remove artifact, noise, and evoked compound potentials, and using bandpass filters 748 specifically tuned to extract frequencies of interest like delta, alpha, beta, gamma, high gamma, or other. The output may be an LFP signal that enhances the sensed oscillation signal, enabling more accurate and detailed feature extraction using the sensed oscillation signal.

FIG. 8 illustrates, by way of example and not limitation, training and use of a machine-learning program. In some example embodiments, machine-learning programs (MLPs), also referred to as machine-learning algorithms or tools, are utilized to perform operations associated with machine learning tasks, such as identifying relationship(s) between detected feature(s) in a sensed biological signal and waveform parameter(s) used to control the neuromodulation. Thus, machine learning may be used to determine the relationships between the extracted features and the stimulation therapy referenced in FIGS. 3 and 323.

Machine learning is a field of study that gives computers the ability to learn without being explicitly programmed. Machine learning explores the study and construction of algorithms that may learn from existing data (e.g., “training data” or “learning data”) and make predictions about new data. Such machine-learning tools may build a model from example training data 849 in order to make data-driven predictions or decisions expressed as outputs or assessments 850. The machine-learning algorithms may use the training data 849 to find correlations among identified features 851 that affect the outcome.

The machine-learning algorithms use features 851 for analyzing the data to generate assessments 850. A feature is an individual measurable property of the observed phenomenon. In the context of a biological signal, some examples of features may include, but are not limited to, peak(s) such as a minimum peak, a maximum peak as well as local minimum and maximum peaks, a range between peaks, a difference in values for features, a feature change with respect to a baseline, an area under a curve, a curve length, an oscillation frequency, and a rate of decay for peak amplitude. Inflection points in the signal may also be an observable feature of the signal, as an inflection point is a point where the signal changes concavity (e.g., from concave up to concave down, or vice versa), and may be identified by determining where the second derivative of the signal is zero. Detected feature(s) may be partially defined by time (e.g., length of curve over a time duration, area under a curve over a time duration, maximum or minimum peak within a time duration, etc.). The features may include time domain features, frequency domain features, or wavelet domain features.

The machine-learning algorithms use the training data 849 to find correlations among the identified features 851 that affect the outcome or assessment 850. With the training data 849 and the identified features 851, the machine-learning tool is trained at operation 852. The machine-learning tool appraises the value of the features 851 as they correlate to the training data 849. The result of the training is the trained machine-learning program 853. Various machine learning techniques may be used to train models to make predictions based on data fed into the models. During a learning phase, the models are developed against a training dataset of inputs to optimize the models to correctly predict the output for a given input. A training data set may be defined for desired functionality of the closed-loop algorithm and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset.

Models may be run against a training dataset for several epochs (e.g., iterations), in which the training dataset is repeatedly fed into the model to refine its results. For example, in a supervised learning phase, a model is developed to predict the output for a given set of inputs, and is evaluated over several epochs to more reliably provide the output that is specified as corresponding to the given input for the greatest number of inputs for the training dataset. In another example, for an unsupervised learning phase, a model is developed to cluster the dataset into n groups, and is evaluated over several epochs as to how consistently it places a given input into a given group and how reliably it produces the n desired clusters across each epoch.

Once an epoch is run, the models are evaluated and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several other machine learning algorithms that may be applied with the present disclosure, including linear regression, neural networks, and the like.

New data 854 is provided as an input to the trained machine-learning program 853, and the trained machine-learning program 853 generates the assessment 850 as output. The outputted assessment 850 may be out of an expected range (e.g., anomalous), indicating that remedial action such as retraining 855 of the machine learning algorithm(s) is warranted. The system also may be configured to determine that the new data 854 includes anomalous data with respect to the training data 849 that was used to train the machine-learning program. The detection of new data that is anomalous may trigger remedial action(s) such as, if it is determined that the previously used training data is outdated, retraining 855 the machine learning program using updated training data.

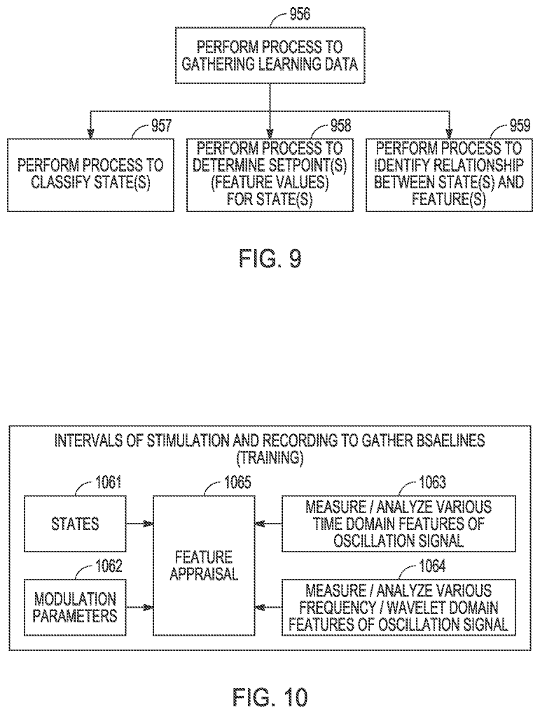

FIG. 9 illustrates, by way of example and not limitation, a process for determining state(s), feature value(s) for state(s) and relationships between state(s) and feature(s) from learning (or training) data. A process may be performed to gather the learning or training data 956. This gathered data may be used to perform processes, including perform a process to classify state(s) 957, perform a process to determine setpoint(s) for the states 958, and/or perform a process to identify relationship(s) between the state(s) and the feature(s) 959. Thus, a closed-loop control algorithm may be implemented to control the therapy using the identified relationship between state(s) and feature(s) and the setpoints (feature values).

FIG. 10 illustrates, by way of example and not limitation, learning data that may be used to provide the machine-learning program training. The setpoints, such as the setpoints illustrated in FIGS. 4 and 5, may represent the “state” of the spinal cord. These spinal cord states may represent a healthy, pain-free condition or a distressed condition associated with worse symptoms or outcomes, as inferred through measured or analyzed oscillations.

Stimulation and recording intervals can be configured for the appropriate gathering of baselines, as illustrated at 1060. The intervals may be based on known stimulation onset/offset times, patient preference, signal duration necessary for processing, and the like. The data gathered for the training data may include spinal cord states 1061, modulation parameters 1062, various time domain features of the oscillation signal 1063, and various frequency or wavelet domain features of the oscillation signal 1064. The states (e.g., spinal cord, peripheral nerve or autonomic nerve states) may be entered by a patient or user (e.g., pain level, therapy rating, etc.), or may be determined as part of supervised, semi-supervised or unsupervised machine learning process. Examples of time domain features that may be measured include, but are not limited to, peak to peak amplitude, standard deviation vs. mean (e.g., DC-offset amplitude, RMS), oscillation frequency, variance of peak-to-peak times, variance of the individual min-max ranges, area under the curve (AUC), curve length, RIVIS amplitude, a regression measure of drift over time, or a measure of power. Examples of frequency domain features that may be measured include, but are not limited to, signal overall power in passband, signal overall power at specific bands, max peaks within specific target bands, width of peak, area under the curve of the peak, curve length between specified values around the peak, or standard deviation of height of X most prominent peaks. the machine learning may train on this data to provide the illustrated feature appraisal 1065. Thus, the spinal cord states, modulation parameters, time domain feature(s) and frequency/wavelet domain feature(s) may provide the training data 849 on which the machine-learning program may be trained (feature appraisal) 852, as illustrated in FIG. 9.

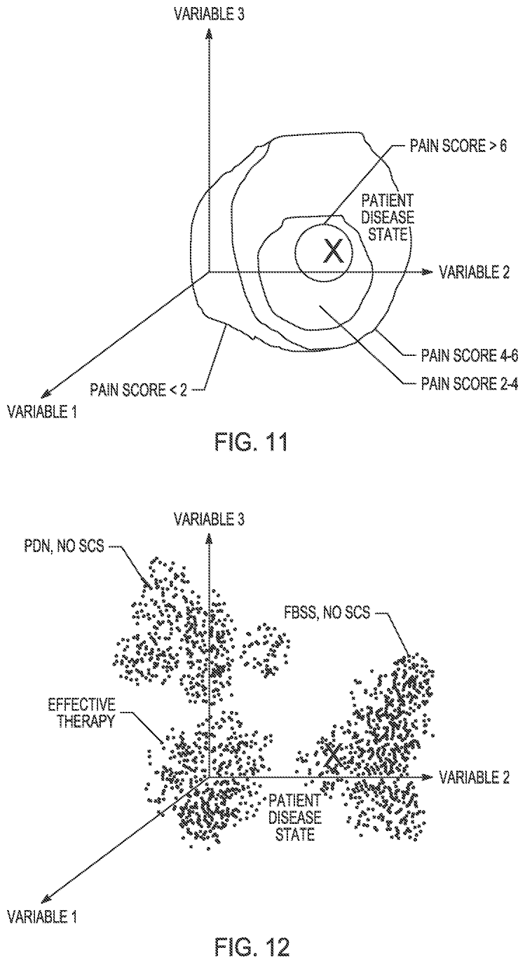

Oscillation characteristics may be indicative of disease and/or dorsal horn function. Oscillation may therefore be classified (i.e., “state” identified) based on its quantitative characteristics. FIG. 11 illustrates, by way of example and not limitation, quantitative mapping of metrics defining oscillations to qualitative state such as patient pain, other symptoms levels, therapy ratings, side-effect ratings, and the like. The figure illustrates the use of three variables to form a three-dimensional space. However, it is noted that the mapping is not limited to three variables/three-dimension spaces. In FIG. 11, the contours may represent patient pain or other symptom levels, therapy ratings, side-effect ratings, and the like. The oscillation characteristics may be correlated with patient reports. FIG. 12 illustrates, by way of example and not limitation, quantitative mapping of metrics defining oscillations overlaid on pre-defined data sets to identify a patient disease state. Similar to FIG. 11, three variables form a three-dimensional space, but the mapping is not limited to three variables/three-dimension spaces. The dots represent patient data points. The colors may represent etiologies/conditions, such as painful diabetic neuropathy (PDN) without SCS therapy, failed back surgery syndrom (FBSS) without SCS therapy, and effective therapy. In this example, the patient states may or may not correlate with patient reports. Thus, “state” may be defined as the quantitative mapping of specific metrics defining the oscillations in question, either stand-alone (e.g., in a “baseline” vs. “therapy”), qualitative (FIG. 11), or overlaid upon pre-defined datasets (FIG. 12). Variables may be pre-defined based on a priori study data and/or selected by using a programmer. The plots illustrated in FIGS. 11 and 12 assume that variable(s) are somewhat independent/uncorrelated with each other.

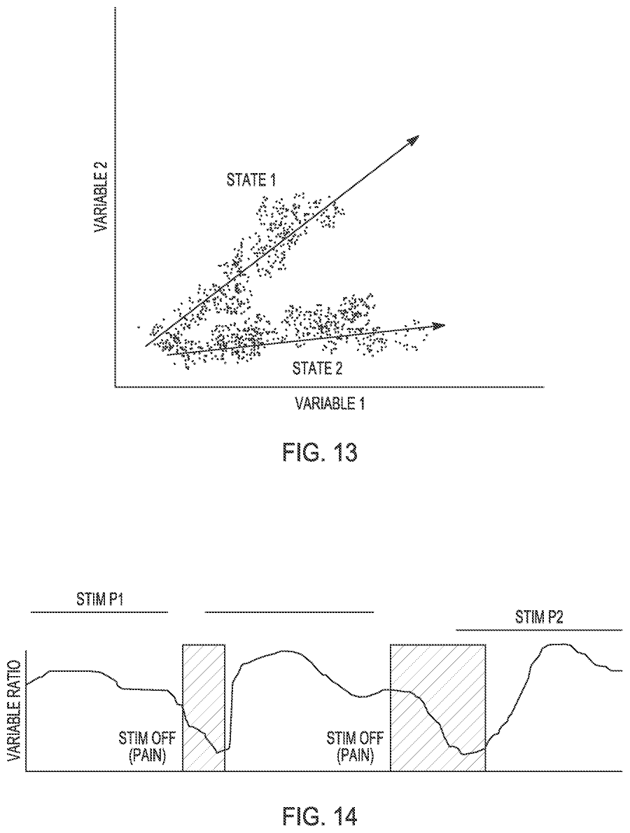

FIG. 13 illustrates, by way of example and not limitation, the use of regression fits to identify relationships between state(s) and some extracted feature(s) from the oscillation signal when the states are correlated. If states are correlated, then relationships between patient state measurement(s) (e.g., on different electrodes, over different amplitudes of stimulation) can be mapped and used as a potential biomarker. Stimulation parameter(s), time, patient satisfaction, and the like can all be included as hidden parametric variables. These fits can be defined by disease state as well, and more than two variables may be plotted.

Dependent or correlated variables may be plotted against the parameter and/or programming settings and used as a more specific control signal. FIG. 14 illustrates, by way of example and not limitation, variables plotted as a ratio against a parameter and/or programming settings (“Stim P1” and Stim P2″), such as may be used as a control signal. All such measurements may be taken not just with a symptom (e.g., pain) but also patient satisfaction (overall), side effect measures, and the like.

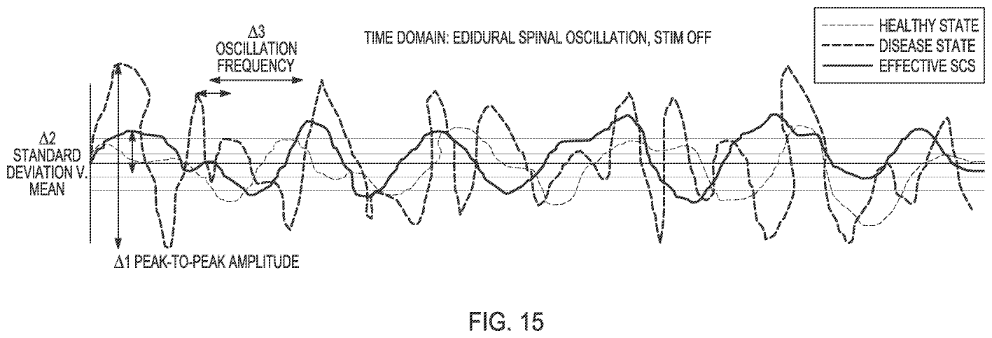

FIG. 15 illustrates, by way of example and not limitation, epidural oscillation signals in the time domain representing a healthy state, a disease state and effective spinal cord stimulation. The illustrated epidural oscillation signals are present when stimulation is off. The illustration indicates that there are observable differences in extracted parameters such as peak-to-peak amplitude, standard deviation, and oscillation frequency for different states. Setpoints, as illustrated in FIGS. 4 and 5, may represent the “state” of the spinal cord, and the spinal cord state may represent a healthy, pain-free condition or a distressed condition associated with worse symptoms or outcomes, as may inferred through measured or analyzed oscillations. Multiple such states and their features can be compared.

The current disease state may be compared against an effective SCS, PNS, ANS to determine dState/dSCS or dState/dPNS, or dState/ANS, which represents a change in a state with respect to a change in an SCS, PNS, or ANS parameter. This analysis can also be performed with frequency domain figures, previously described. The system may be configured to measure differences in a disease state vs. effective stimulation oscillations and state and/or healthy state to establish a variable “vector” field. A simple example is plotting peak-to-peak voltage difference versus an oscillation frequency difference as a function of stimulation amplitude.

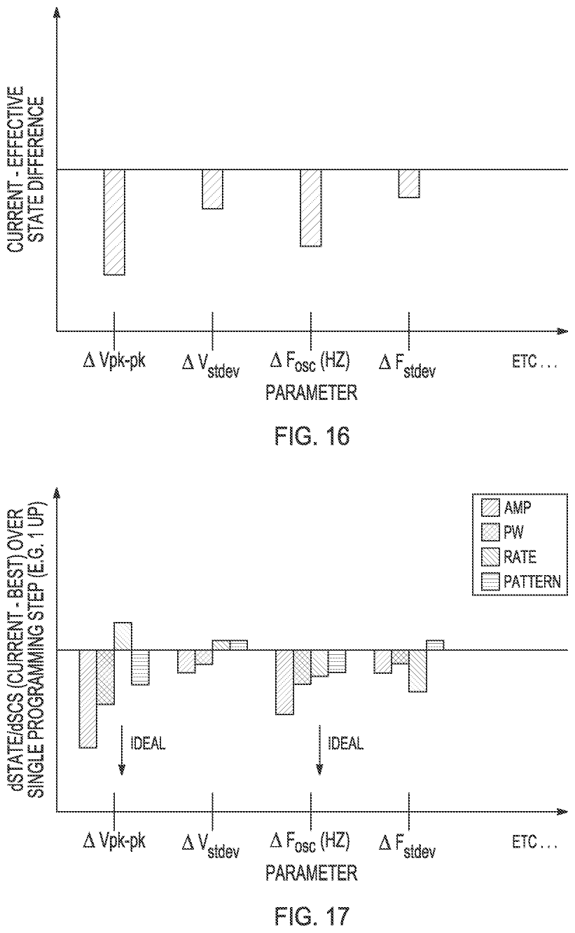

FIG. 16 illustrates a sensitivity plot identifying parameter(s) most or least different between good and bad states. Standard methods may be used to optimize. The sensitivity analysis may also be performed in a frequency domain. In the illustrated embodiment, the peak-to-peak voltage shows the most difference between good and bad states, followed by the oscillation frequency. Therefore, in this illustration, the peak-to-peak voltage and oscillation frequency may be the extracted features selected for use to the ability of various stimulation parameters to modulate the extracted features.

FIG. 17 illustrates an example in which the stimulation parameters (amplitude, pulse width, frequency or rate, and pattern) are assessed for their ability to modulate the extracted feature(s) determined to be most sensitive to changes between the good and bad states. The best parameter(s), based on this assessment, may be selected for control. More particularly, the stimulation parameters may be evaluated to determine which stimulation parameters correspond to the largest differences (or smallest differences) between “good” and “bad states” in the peak-to-peak voltage and oscillation frequency. The controller may pick the parameter that creates biggest change towards “ideal” direction for each parameter. In the illustrated case, the amplitude stimulation parameter moves selected states best from the current state to the ideal state. Thus, amplitude may be selected for control. This analysis may be performed in the frequency domain. Some embodiments may perform the analysis in both the time domain and the frequency domain.

Any of dState/dSCS, or dState/dPNS, or dState/dANS can be mapped over larger range, and a standard control, such as PID or Kalman, may be used to modulate the therapy. Other states could be ignored, weighted, or included as previously mentioned. If there is a conflict, the parameter that provides the best “net” change, the best weighted change, and/or that produces the greatest change in the most important state metric is used. Other parameters can also be used a “tiebreakers.”

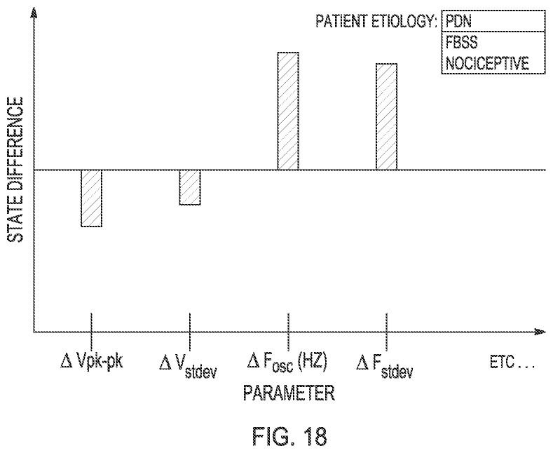

FIG. 18 illustrates, by way of example and not limitation, selection of a parameter for use in controlling the therapy based on a-priori knowledge of disease state. The illustrated examples of patient etiology include painful diabetic neuropathy (PDN), failed back surgery syndrome (FBSS), and Nociceptive pain. Other patient etiologies may be used. In the illustrated embodiment in which PDN is selected, the controller may select the oscillation frequency and the standard deviation for frequency as the parameters for use to define the state or setpoint for PDN, as they exhibit the largest changes for changes in states. The analysis can also include and/or be performed in frequency domain. It is noted that the illustrated etiologies and selected parameter(s) is merely an example of selecting specific parameters based on specific etiology, and is not intended indicate that these parameters should be used to set the state or setpoint for PDN.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering a neuromodulation signal according to neuromodulation parameters to neural tissue;
sensing local field potentials within a spinal cord or a peripheral nerve indicative of oscillations;
extracting one or more features from the local field potentials that are indicative of the oscillations;
providing a comparison of the extracted one or more feature(s) to corresponding one or more setpoints; and
controlling the delivery of the neuromodulation signal based on the comparison.

2. The method of claim 1, wherein the extracting one or more features includes extracting at least one of a time domain feature, a frequency domain feature or a wavelet domain feature.

3. The method of claim 1, wherein the extracting one or more features include the extracting at least one time domain feature.

4. The method of claim 3, wherein the extracting at least one time domain feature includes extracting at least one of: peak to peak amplitude, standard deviation vs. mean, oscillation frequency, variance of peak-to-peak times, variance of the individual min-max ranges, area under the curve (AUC), curve length, RMS amplitude, a regression measure of drift over time, or a measure of power.

5. The method of claim 1, wherein the extracting one or more features includes the extracting at least one frequency domain feature.

6. The method of claim 5, wherein the extracting at least one frequency domain feature includes extracting at least one of: signal overall power in passband or at specific bands, max peaks within specific target bands, width of peak, standard deviation of height of X most prominent peaks, or an area under a curve of a maximum peak.

7. The method of claim 1, wherein the corresponding setpoint(s) includes at least one feature for the local field potentials indicative of oscillations corresponding to a symptom level, a therapy rating or side-effect ratings.

8. The method of claim 1, wherein the controlling the delivery of the neuromodulation signal based on the comparison includes providing a feedback closed loop control using a Proportion Integral Derivative (PID), PID with thresholds, a lookup table, a Kalman control, an On/Off control or a threshold control.

9. The method of claim 1, wherein the sensing local field potentials indicative of spinal cord oscillations includes using electrodes designed, placed or orientated to enhance sensing of spinal cord oscillations.

10. The method of claim 9, wherein the electrodes include:
electrodes arranged in a paddle array and rostrocaudally orientated;
cylindrical electrodes with a large diameter or large surface area to increase sensing surface;
intradural electrodes;
epidural electrodes; or
electrodes placed over the dorsal horn.

11. The method of claim 1, further comprising:
filtering the sensed local field potentials to filter out at least one of noise, ECAPs, or one or more artifacts; or
performing bandpass filtering frequencies of interest.

12. The method of claim 1, further comprising using at least one other sensor to provide at least one other sensor signal, extracting at least one other feature from the other sensor signal, and providing a comparison to the at least one other feature from the other sensor signal to at least one other setpoint.

13. The method of claim 1, further comprising using machine learning to evaluate learning data to classify the one or more setpoints.

14. The method of claim 13, wherein the using machine learning includes using a neural network, a Support Vector Machine (SVM), a least square model, or a mean squares model to determine how state variables change with stimulation, for use in controlling the delivery of the neuromodulation signal.

15. The method of claim 1, further comprising gathering learning data using intervals of stimulation and recording, wherein the intervals between stimulation are determined using known stimulation onset/offset times, patient preference, or a signal duration of sufficient length or with sufficient delay to obtain a desired amount of learning data with appropriate delay.

16. The method of claim 1, wherein the one or more setpoints correspond to a state based on a quantitative mapping of features for the local field potentials that are indicative of the spinal cord oscillations for: baseline and therapy; qualitative based on a pain score; or overlaid upon pre-defined datasets.

17. The method of claim 1, further comprising mapping relationships between correlated features and states, wherein the mapping relationships includes:
performing a regressive fit for each state to determine the relationships between the corresponding state and features; or
plotting a ratio of correlated variables against programming parameters.

18. The method of claim 1, further comprising:
comparing multiple states and their features to determine dState/(dSCS or dPNS or dANS), where dState/(dSCS or dPNS or dANS) represents a change in a state with respect to a change in an spinal cord stimulation (SCS) parameter or a peripheral nerve stimulation (PNS) parameter or an autonomic nerve stimulation (ANS) parameter; and
performing a sensitivity analysis on a plurality of parameters to identify one or more of the plurality of parameters having a greater change between bad and good states with a change in SCS, PNS or ANS parameter than other ones of the plurality of parameters,
wherein the controlling the delivery of the neuromodulation signal based on the comparison includes modulating the one or more of the plurality of parameters based on the comparison.

19. The method of claim 1, wherein the one or more setpoints corresponds to a preconfigured state determined based on a patient's diagnosis or other demographic factors, or correspond to a user-customizable state.

20. A non-transitory machine-readable medium including instructions, which when executed by a machine, cause the machine to perform a method comprising:
delivering a neuromodulation signal according to neuromodulation parameters to neural tissue;
sensing local field potentials within a spinal cord or a peripheral nerve indicative of oscillations;
extracting one or more features for the local field potentials that are indicative of the oscillations;
providing a comparison of the extracted one or more features to corresponding one or more setpoints; and
controlling the delivery of the neuromodulation signal based on the comparison.
